(19) United States
(12) Patent Application Publication
(10) Pub. No.: US 2024/0197767 A1
(43) Pub. Date: Jun. 20, 2024

(54) COMBINATION OF ZIDOVUDINE WITH A TETRACYCLINE ANTIBIOTIC

(71) Applicant: Helperby Therapeutics Limited, London Greater London (GB)

(72) Inventors: Professor Anthony Coates, London Greater London (GB); Yanmin Hu, London Greater London (GB)

(73) Assignee: Helperby Therapeutics Limited, London Greater London (GB)

(21) Appl. No.: 17/768,669

(22) PCT Filed: Oct. 9, 2020
(86) PCT No.: PCT/GB2020/052525

§ 371 Date: Apr. 13, 2022

(30) Foreign Application Priority Data
Oct. 14, 2019 (GB) 1914847.7

(51) Int. Cl. A61K 31/7072; A61K 31/65; A61P 31/04

(52) U.S. Cl. CPC A61K 31/7072; A61K 31/65; A61P 31/04

(57) ABSTRACT

The present invention relates to a combination comprising zidovudine or a pharmaceutically acceptable derivative thereof and a tetracycline antibiotic or a pharmaceutically acceptable derivative or prodrug thereof, for use in treating a microbial infection, particularly a bacterial infection such as a urinary tract infection.

### FIELD OF THE INVENTION

The present invention relates to the combination of zidovudine or a pharmaceutically acceptable derivative thereof with a tetracycline antibiotic compound or a pharmaceutically acceptable derivative or prodrug thereof selected from the group defined herein, for use in the treatment of microbial infections. In particular, it relates to the use of such combinations to kill multiplying (i.e. log phase) microorganisms associated with microbial infections, e.g. Gram-negative bacterial infections including urinary tract infections.

### BACKGROUND

Before the introduction of antibiotics, patients suffering from acute microbial infections (e.g. tuberculosis or pneumonia) had a low chance of survival. For example, mortality from tuberculosis was around 50%. Although the introduction of antimicrobial agents in the 1940s and 1950s rapidly changed this picture, bacteria have responded by progressively gaining resistance to commonly used antibiotics. Now, every country in the world has antibiotic-resistant bacteria.

Indeed, more than 70% of bacteria that give rise to hospital acquired infections in the USA resist at least one of the main antimicrobial agents that are typically used to fight infection (Nature Reviews, Drug Discovery, 1, 895-910 (2002)). The World Health Organization has therefore classified antimicrobial resistance as a “serious threat [that] is no longer a prediction for the future, it is happening right now in every region of the world and has the potential to affect anyone, of any age, in any country” (“Antimicrobial resistance: global report on surveillance”, The World Health Organization, April 2014).

One group of antibiotics which is facing critical resistance problems is the compounds used to treat urinary tract infections (UTIs) and specifically genitourinary infections. In a recent report from Public Health England, it was noted that antimicrobial resistance was common in more than 1 million urinary tract infections caused by bacteria identified in NHS laboratories in 2016 («English Surveillance Programme for Antimicrobial Utilisation and Resistance (ESPAUR)» (2017)).

A solution to the growing problem of resistant bacteria causing urinary tract infections is therefore desperately needed.

WO2014/147405 describes the use of zidovudine in combination with a polymyxin selected from colistin and polymyxin B for treating a microbial infection. WO2015/114340 describes the use of zidovudine in combination with a polymyxin selected from colistin or polymyxin B, an anti-tuberculosis antibiotic selected from rifampicin, rifapentine or rifabutin and optionally piperine, for treating a microbial infection. WO2018/011562 describes a combination comprising zidovudine and a carbapenem, optionally with a polymyxin selected from polymyxin B and polymyxin E.

### SUMMARY OF THE INVENTION

Surprisingly and of huge importance to the fight against antimicrobial resistance in the treatment of urinary tract infections, the Applicant has discovered that the antiretroviral drug zidovudine has a synergistic effect with tetracycline antibiotics. In other words, the combination(s) has a greater biological activity than the expected additive effect of each agent at the stated dosage level.

Zidovudine (AZT) is a nucleoside analogue reverse-transcriptase inhibitor, a type of antiretroviral drug which is used for the treatment of HIV/AIDS infection. As well as its antiretroviral activity, the antibacterial effect of zidovudine (AZT) has been demonstrated both in vitro and in vivo with experimental models of gram-negative bacteria infections (Hermann et al., Antimicrob Agents Chemther. 1992 May; 36(5): 1081-1085). There have also been reports of zidovudine being active as an anti-microbial when combined with the antibiotic gentamicin (Doléans-Jordheim A. et al., Eur J Clin Microbiol Infect Dis. 2011 October; 30(10):1249-56).

Synergy is not predictable or expected when two actives are used in combination. The present invention is therefore based on the unexpected finding that zidovudine or a pharmaceutically acceptable derivative thereof exhibits synergistic antimicrobial activity when used in combination with a tetracycline antibiotic compound or a pharmaceutically acceptable derivative or prodrug thereof, against log phase (i.e. multiplying) microorganisms. Notably synergy is seen when the combinations are used against gram-negative bacteria.

The surprising biological activity of the combinations of the present invention offers the opportunity to rejuvenate certain urinary tract antibiotics, against which bacterial resistance has developed.

Synergy in the context of antimicrobial drugs is measured in a number of ways that conform to the generally accepted opinion that “synergy is an effect greater than additive”. One of the ways to assess whether synergy has been observed is to use the “chequerboard” technique. This is a well-accepted method that leads to the generation of a value called the fractional inhibitory concentration index (FICI). Orhan et al., J. Clin. Microbiol. 2005, 43(1):140 describes the chequerboard method and analysis in the paragraph bridging pages 140-141, and explains that the FICI value is a ratio of the sum of the MIC (Minimum Inhibitory Concentration) level of each individual component alone and in the mixture. The combination is considered synergistic when the ΣFIC is ≤0.5, indifferent when the ΣFIC is >0.5 but <4.0, and antagonistic when the ΣFIC is >4.0.

Another accepted test for ascertaining the presence or absence of synergy is to use time-kill methods. This involves the dynamic effect of a drug combination being compared to each drug alone when assessing the effect on bacterial log or stationary-growth over time. Again, the possible results are for synergistic, additive or antagonistic effects.

In one aspect the present invention provides a combination of zidovudine or a pharmaceutically acceptable derivative thereof and a tetracycline antibiotic or a pharmaceutically acceptable derivative or prodrug thereof, for use in the treatment of a microbial infection.

In another aspect the present invention provides the use of zidovudine or a pharmaceutically acceptable derivative thereof in combination with a tetracycline antibiotic or a pharmaceutically acceptable derivative or prodrug thereof, in the manufacture of a medicament for treating a microbial infection.

In another aspect the present invention provides the use of a tetracycline antibiotic or a pharmaceutically acceptable derivative or prodrug thereof, in combination with zidovudine or a pharmaceutically acceptable derivative thereof, in the manufacture of a medicament for treating a microbial infection.

In a further aspect, the invention provides a method of treating a microbial infection which comprises administering to a mammal, including man, zidovudine or a pharmaceutically acceptable derivative thereof in combination with a tetracycline antibiotic or a pharmaceutically acceptable derivative or prodrug thereof.

In each of the above aspects, the tetracycline antibiotic may be selected from the group consisting of tetracycline, chlortetracycline, oxytetracycline, demeclocycline, lymecycline, meclocycline, methacycline, minocycline, rolitetracycline, doxycycline, tigecycline, eravacycline, sarecycline, omadacycline, and pharmaceutically acceptable derivatives and prodrugs thereof. In preferred embodiments of each of the above aspects, the tetracycline antibiotic is selected from the group consisting of tetracycline, chlortetracycline, oxytetracycline, demeclocycline, meclocycline, methacycline, doxycycline, and pharmaceutically acceptable derivatives and prodrugs thereof. In more preferred embodiments, the tetracycline antibiotic is selected from the group consisting of tetracycline, chlortetracycline, oxytetracycline, demeclocycline, doxycycline, and pharmaceutically acceptable derivatives and prodrugs thereof. In even more preferred embodiments, the tetracycline antibiotic is selected from the group consisting of tetracycline, chlortetracycline, oxytetracycline, doxycycline, and pharmaceutically acceptable derivatives and prodrugs thereof. In the most preferred embodiment, the tetracycline antibiotic is doxycycline or a pharmaceutically acceptable derivative or prodrug thereof.

There is also provided a pharmaceutical composition comprising zidovudine or a pharmaceutically acceptable derivative thereof in combination with a tetracycline antibiotic or a pharmaceutically acceptable derivative or prodrug thereof, and a pharmaceutically acceptable adjuvant, diluent or carrier, for use in the treatment of a microbial infection, preferably wherein the microbial infection is a bacterial infection, e.g. a gram-negative bacterial infection. The tetracycline antibiotic may be defined as above.

In a further aspect, the invention relates to a product comprising zidovudine or a pharmaceutically acceptable derivative thereof and a tetracycline antibiotic or a pharmaceutically acceptable derivative or prodrug thereof, as a combined preparation for simultaneous, separate or sequential use in killing multiplying microorganisms associated with a microbial infection. Preferably for killing multiplying bacteria associated with a bacterial infection, e.g. a gram-negative bacterial infection. The tetracycline antibiotic may be as defined above for the other aspects of the present invention.

### BRIEF DESCRIPTION OF THE FIGURES

FIG. 1 is a chequerboard from the Examples showing synergy testing of zidovudine and doxycycline on BAA2469 NDM-1 Escherichia coli. Compound concentrations are provided in mg/L.

FIG. 2 is a chequerboard from the Examples showing synergy testing of zidovudine and doxycycline on BAA2470 NDM-1 Klebsiella pneumoniae. Compound concentrations are provided in mg/L.

FIG. 3 is a chequerboard from the Examples showing synergy testing of zidovudine and doxycycline on BAA2471 NDM-1 Escherichia coli. Compound concentrations are provided in mg/L.

FIG. 4 is a chequerboard from the Examples showing synergy testing of zidovudine and doxycycline on BAA2472 NDM-1 Klebsiella pneumoniae. Compound concentrations are provided in mg/L.

FIG. 5 is a chequerboard from the Examples showing synergy testing of zidovudine and doxycycline on NCTC13443 NDM-1 Klebsiella pneumoniae. Compound concentrations are provided in mg/L.

### DETAILED DESCRIPTION OF THE INVENTION

As used herein, the expressions “combination of” and “in combination with” cover separate, sequential and simultaneous administration of the agents. Unless specified to the contrary, the expressions are also intended to exclude any additional actives, e.g. “a combination of zidovudine and doxycycline” means that zidovudine and doxycycline are administered separately, sequentially or simultaneously but that no other actives are administered.

When the agents are administered sequentially, either the zidovudine or the tetracycline antibiotic compound may be administered first. When administration is simultaneous, the agents may be administered either in the same or a different pharmaceutical composition. Adjunctive therapy, i.e. where one agent is used as a primary treatment and the other agent(s) is used to assist that primary treatment, is also an embodiment of the present invention.

The combinations of the present invention may be used to treat microbial infections. In particular they may be used to kill multiplying and/or clinically latent microorganisms associated with microbial infections, preferably multiplying microorganisms associated with microbial infections, e.g. multiplying bacteria associated with Gram-negative bacterial infections.

References herein to the treatment of a microbial infection therefore include killing multiplying and/or clinically latent microorganisms associated with such infections.

As used herein, “kill” means a loss of viability as assessed by a lack of metabolic activity.

As used herein, “clinically latent microorganism” means a microorganism that is metabolically active but has a growth rate that is below the threshold of infectious disease expression. The threshold of infectious disease expression refers to the growth rate threshold below which symptoms of infectious disease in a host are absent.

The metabolic activity of clinically latent microorganisms can be determined by several methods known to those skilled in the art; for example, by measuring mRNA levels in the microorganisms or by determining their rate of uridine uptake. In this respect, clinically latent microorganisms, when compared to microorganisms under logarithmic growth conditions (in vitro or in vivo), possess reduced but still significant levels of:
(1) mRNA (e.g. from 0.0001 to 50%, such as from 1 to 30, 5 to 25 or 10 to 20%, of the level of mRNA); and/or
(II) uridine (e.g. [³H]uridine) uptake (e.g. from 0.0005 to 50%, such as from 1 to 40, 15 to 35 or 20 to 30% of the level of [³H]uridine uptake).

Clinically latent microorganisms typically possess a number of identifiable characteristics. For example, they may be viable but non-culturable; i.e. they cannot typically be detected by standard culture techniques, but are detectable and quantifiable by techniques such as broth dilution counting, microscopy, or molecular techniques such as polymerase chain reaction. In addition, clinically latent microorganisms are phenotypically tolerant, and as such are sensitive (in log phase) to the biostatic effects of conventional antimicrobial agents (i.e. microorganisms for which the minimum inhibitory concentration (MIC) of a conventional antimicrobial is substantially unchanged); but possess drastically decreased susceptibility to drug-induced killing (e.g. microorganisms for which, with any given conventional antimicrobial agent, the ratio of minimum microbiocidal concentration (e.g. minimum bactericidal concentration, MBC) to MIC is 10 or more).

As used herein, the term “microorganisms” means fungi and bacteria. References herein to “microbial”, “antimicrobial” and “antimicrobially” shall be interpreted accordingly. For example, the term “microbial” means fungal or bacterial, and “microbial infection” means any fungal or bacterial infection.

In various embodiments of the invention, one or more of the aforementioned combinations is used to treat a bacterial infection, in particular the combinations may be used to kill multiplying and/or clinically latent microorganisms associated with a bacterial infection. Preferably multiplying bacteria associated with a bacterial infection. As used herein, the term “bacteria” (and derivatives thereof, such as “microbial infection”) includes, but is not limited to, references to organisms (or infections due to organisms) of the following classes and specific types: Gram-positive cocci, such as Staphylococci (e.g. Staph. aureus, Staph. epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus); Streptococci (e.g. beta-haemolytic, pyogenic streptococci (such as Strept. agalactiae, Strept. canis, Strept. dysgalactiae dysgalactiae, Strept. dysgalactiae equisimilis, Strept. equi equi, Strept. equi zooepidemicus, Strept. iniae, Strept. porcinus and Strept. pyogenes), microaerophilic, pyogenic streptococci (Streptococcus “milleri”, such as Strept. anginosus, Strept. constellatus constellatus, Strept. constellatus pharyngidis and Strept. intermedius), oral streptococci of the “mitis” (alpha-haemolytic—Streptococcus “viridans”, such as Strept. mitis, Strept. oralis, Strept. sanguinis, Strept. cristatus, Strept. gordonii and Strept. parasanguinis), “salivarius” (non-haemolytic, such as Strept. salivarius and Strept. vestibularis) and “mutans” (tooth-surface streptococci, such as Strept. criceti, Strept. mutans, Strept. ratti and Strept. sobrinus) groups, Strept. acidominimus, Strept. bovis, Strept. faecalis, Strept. equinus, Strept. pneumoniae and Strept. suis, or Streptococci alternatively classified as Group A, B, C, D, E, G, L, P, U or V Streptococcus); Gram-negative cocci, such as Neisseria gonorrhoeae, Neisseria meningitidis, Neisseria cinerea, Neisseria elongata, Neisseria flavescens, Neisseria lactamica, Neisseria mucosa, Neisseria sicca, Neisseria subflava and Neisseria weaveri; Bacillaceae, such as Bacillus anthracis, Bacillus subtilis, Bacillus thuringiensis, Bacillus stearothermophilus and Bacillus cereus; Enterobacteriaceae, such as Escherichia coli, Enterobacter (e.g. Enterobacter aerogenes, Enterobacter agglomerans and Enterobacter cloacae), Citrobacter (such as Citrob. freundii and Citrob. divernis), Hafnia (e.g. Hafnia alvel), Erwinia (e.g. Erwinia persicinus), Morganella morganii, Salmonella (Salmonella enterica and Salmonella typhi), Shigella (e.g. Shigella dysenteriae, Shigella flexneri, Shigella boydii and Shigella sonnei), Klebsiella (e.g. Klebs. pneumoniae, Klebs. oxytoca, Klebs. ornitholytica, Klebs. planticola, Klebs. ozaenae, Klebs. terrigena, Klebs. granulomatis (Calymmatobacterium granulomatis) and Klebs. rhinoscleromatis), Proteus (e.g. Pr. mirabilis, Pr. rettgeri and Pr. vulgaris), Providencia (e.g. Providencia alcalifaciens, Providencia rettgeri and Providencia stuartih), Serratia (e.g. Serratia marcescens and Serratia liquifaciens), and Yersinia (e.g. Yersinia enterocolitica, Yersinia pestis and Yersinia pseudotuberculosis); Enterococci (e.g. Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus and Enterococcus solitarius); Helicobacter (e.g. Helicobacter pylori, Helicobacter cinaedi and Helicobacter fennelliae); Acinetobacter (e.g. A. baumanii, A. calcoaceticus, A. haemolyticus, A. johnsonii, A. junii, A. lwoffi and A. radioresistens); Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzen); Bacteroides fragilis; Peptococcus (e.g. Peptococcus niger); Peptostreptococcus; Clostridium (e.g. C. perfringens, C. difficile, C. botulinum, C. tetani, C. absonum, C. argentinense, C. baratii, C. bifermentans, C. beijerinckii, C. butyricum, C. cadaveris, C. carnis, C. celatum, C. clostridioforme, C. cochlearium, C. cocleatum, C. fallax, C. ghonii, C. glycolicum, C. haemolyticum, C. hastiforme, C. histolyticum, C. indolis, C. innocuum, C. irregulare, C. leptum, C. limosum, C. malenominatum, C. novyi, C. oroticum, C. paraputrificum, C. piliforme, C. putrefasciens, C. ramosum, C. septicum, C. sordelii, C. sphenoides, C. sporogenes, C. subterminale, C. symbiosum and C. tertium); Mycoplasma (e.g. M. pneumoniae, M. hominis, M. genitalium and M. urealyticum); Mycobacteria (e.g. Mycobacterium tuberculosis, Mycobacterium avium, Mycobacterium fortuitum, Mycobacterium marinum, Mycobacterium kansasii, Mycobacterium chelonae, Mycobacterium abscessus, Mycobacterium leprae, Mycobacterium smegmitis, Mycobacterium africanum, Mycobacterium alvei, Mycobacterium asiaticum, Mycobacterium aurum, Mycobacterium bohemicum, Mycobacterium bovis, Mycobacterium branderi, Mycobacterium brumae, Mycobacterium celatum, Mycobacterium chubense, Mycobacterium confluentis, Mycobacterium conspicuum, Mycobacterium cookii, Mycobacterium flavescens, Mycobacterium gadium, Mycobacterium gastri, Mycobacterium genavense, Mycobacterium gordonae, Mycobacterium goodii, Mycobacterium haemophilum, Mycobacterium hassicum, Mycobacterium intracellulare, Mycobacterium interjectum, Mycobacterium heidelberense, Mycobacterium lentiflavum, Mycobacterium malmoense, Mycobacterium microgenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium szulgai, Mycobacterium terrae, Mycobacterium thermoresistabile, Mycobacterium triplex, Mycobacterium triviale, Mycobacterium tusciae, Mycobacterium ulcerans, Mycobacterium vaccae, Mycobacterium wolinskyi and Mycobacterium xenopi); Haemophilus (e.g. Haemophilus influenzae, Haemophilus ducreyi, Haemophilus aegyptius, Haemophilus parainfluenzae, Haemophilus haemolyticus and Haemophilus parahaemolyticus); Actinobacillus (e.g. Actinobacillus actinomycetemcomitans, Actinobacillus equuli, Actinobacillus hominis, Actinobacillus lignieresii, Actinobacillus suis and Actinobacillus ureae); Actinomyces (e.g. Actinomyces israelii); Brucella (e.g. Brucella abortus, Brucella canis, Brucella melintensis and Brucella suis); Campylobacter (e.g. Campylobacter jejuni, Campylobacter coli, Campylobacter lari and Campylobacter fetus); Listeria monocytogenes; Vibrio (e.g. Vibrio cholerae and Vibrio parahaemolyticus, Vibrio alginolyticus, Vibrio carchariae, Vibrio fluvialis, Vibrio furnissii, Vibrio hollisae, Vibrio metschnikovii, Vibrio mimicus and Vibrio vulnificus); Erysipelothrix rhusiopathiae; Corynebacteriaceae (e.g. Corynebacterium diphtheriae, Corynebacterium jeikeum and Corynebacterium urealyticum); Spirochaetaceae, such as Borrelia (e.g. Borrelia recurrentis, Borrelia burgdorferi, Borrelia afzelii, Borrelia andersonii, Borrelia bissettii, Borrelia garinii, Borrelia japonica, Borrelia lusitaniae, Borrelia tanukii, Borrelia turdi, Borrelia valaisiana, Borrelia caucasica, Borrelia crocidurae, Borrelia duttoni, Borrelia graingeri, Borrelia hermsii, Borrelia hispanica, Borrelia latyschewii, Borrelia mazzottii, Borrelia parkeri, Borrelia persica, Borrelia turicatae and Borrelia venezuelensis) and Treponema (Treponema pallidum ssp. pallidum, Treponema pallidum ssp. endemicum, Treponema pallidum ssp. pertenue and Treponema carateum); Pasteurella (e.g. Pasteurella aerogenes, Pasteurella bettyae, Pasteurella canis, Pasteurella dagmatis, Pasteurella gallinarum, Pasteurella haemolytica, Pasteurella multocida multocida, Pasteurella multocida gallicida, Pasteurella multocida septica, Pasteurella pneumotropica and Pasteurella stomatis); Bordetella (e.g. Bordetella bronchiseptica, Bordetella hinzii, Bordetella holmseii, Bordetella parapertussis, Bordetella pertussis and Bordetella trematum); Nocardiaceae, such as Nocardia (e.g. Nocardia asteroides and Nocardia brasiliensis); Rickettsia (e.g. Ricksettsii or Coxiella burnetih); Legionella (e.g. Legionalla anisa, Legionalla birminghamensis, Legionalla bozemanii, Legionalla cincinnatiensis, Legionalla dumoffii, Legionalla feeleii, Legionalla gormanii, Legionalla hackeliae, Legionalla israelensis, Legionalla jordanis, Legionalla lansingensis, Legionalla longbeachae, Legionalla maceachernii, Legionalla micdadei, Legionalla oakridgensis, Legionalla pneumophila, Legionalla sainthelensi, Legionalla tucsonensis and Legionalla wadsworthii); Moraxella catarrhalis; Cyclospora cayetanensis; Entamoeba histolytica; Giardia lamblia; Trichomonas vaginalis; Toxoplasma gondii; Stenotrophomonas maltophilia; Burkholderia cepacia; Burkholderia mallei and Burkholderia pseudomallei; Francisella tularensis; Gardnerella (e.g. Gardneralla vaginalis and Gardneralla mobiluncus); Streptobacillus moniliformis; Flavobacteriaceae, such as Capnocytophaga (e.g. Capnocytophaga canimorsus, Capnocytophaga cynodegmi, Capnocytophaga gingivalis, Capnocytophaga granulosa, Capnocytophaga haemolytica, Capnocytophaga ochracea and Capnocytophaga sputigena); Bartonella (Bartonella bacilliformis, Bartonella clarridgeiae, Bartonella elizabethae, Bartonella henselae, Bartonella quintana and Bartonella vinsonii arupensis); Leptospira (e.g. Leptospira biflexa, Leptospira borgpetersenii, Leptospira inadai, Leptospira interrogans, Leptospira kirschneri, Leptospira noguchii, Leptospira santarosai and Leptospira weilh); Spirillum (e.g. Spirillum minus); Bacteroides (e.g. Bacteroides caccae, Bacteroides capillosus, Bacteroides coagulans, Bacteroides distasonis, Bacteroides eggerthii, Bacteroides forsythus, Bacteroides fragilis, Bacteroides merdae, Bacteroides ovatus, Bacteroides putredinis, Bacteroides pyogenes, Bacteroides splanchinicus, Bacteroides stercoris, Bacteroides tectus, Bacteroides thetaiotaomicron, Bacteroides uniformis, Bacteroides ureolyticus and Bacteroides vulgatus); Prevotella (e.g. Prevotella bivia, Prevotella buccae, Prevotella corporis, Prevotella dentalis (Mitsuokella dentalis), Prevotella denticola, Prevotella disiens, Prevotella enoeca, Prevotella heparinolytica, Prevotella intermedia, Prevotella loeschii, Prevotella melaninogenica, Prevotella nigrescens, Prevotella oralis, Prevotella oris, Prevotella oulora, Prevotella tannerae, Prevotella venoralis and Prevotella zoogleoformans); Porphyromonas (e.g. Porphyromonas asaccharolytica, Porphyromonas cangingivalis, Porphyromonas canoris, Porphyromonas cansulci, Porphyromonas catoniae, Porphyromonas circumdentaria, Porphyromonas crevioricanis, Porphyromonas endodontalis, Porphyromonas gingivalis, Porphyromonas gingivicanis, Porphyromonas levii and Porphyromonas macacae); Fusobacterium (e.g. F. gonadiaformans, F. mortiferum, F. naviforme, F. necrogenes, F. necrophorum necrophorum, F. necrophorum fundiliforme, F. nucleatum nucleatum, F. nucleatum fusiforme, F. nucleatum polymorphum, F. nucleatum vincentii, F. periodonticum, F. russii, F. ulcerans and F. varium); Chlamydia (e.g. Chlamydia trachomatis); Cryptosporidium (e.g. C. parvum, C. hominis, C. canis, C. felis, C. meleagridis and C. muris); Chlamydophila (e.g. Chlamydophila abortus (Chlamydia psittaci), Chlamydophila pneumoniae (Chlamydia pneumoniae) and Chlamydophila psittaci (Chlamydia psittaci)); Leuconostoc (e.g. Leuconostoc citreum, Leuconostoc cremoris, Leuconostoc dextranicum, Leuconostoc lactis, Leuconostoc mesenteroides and Leuconostoc pseudomesenteroides); Gemella (e.g. Gemella bergeri, Gemella haemolysans, Gemella morbillorum and Gemella sanguinis); and Ureaplasma (e.g. Ureaplasma parvum and Ureaplasma urealyticum).

Preferably, the bacterial infections treated by the combinations described herein are Gram-negative bacterial infections. Particular Gram-negative bacteria that may be treated using a combination of the invention include: Enterobacteriaceae, such as Escherichia coli, Klebsiella (e.g. Klebs. pneumoniae and Klebs. oxytoca) and Proteus (e.g. Pr. mirabilis, Pr. rettgeri and Pr. vulgaris); Haemophilis influenzae; Mycobacteria, such as Mycobacterium tuberculosis; and Enterobacter (e.g. Enterobacter cloacae). Preferably, the bacteria are Enterobacteriaceae, such as Escherichia coli and Klebsiella (e.g. Klebs. pneumoniae and Klebs. oxytoca). Particularly preferred are Escherichia coli, and Klebs. pneumoniae (e.g. Klebs. pneumoniae subsp. pneumoniae).

In all embodiments it is preferable that the combination therapy is synergistic as compared to the administration of the combination components taken alone.

The combination of the present invention is particularly beneficial in treating (multi)-drug-resistant ((M)DR) bacteria. With respect to Enterobacteriaceae, drug resistance most often builds up to carbapenemase i.e. carbapenemase-resistant strains and “extended spectrum β-lactamase”(ESBL) strains for example New Delhi Metallo-beta-lactamase-1 (NDM-1) resistant Klebs. pneumonia, and NDM-1 E. coli.

It should be kept in mind that although a combination such as that claimed may initially be demonstrated to be functional in treating (M)DR strains, they can then be used in treating non-resistant strains. This is especially valuable in the context of the presently claimed combination where the primary therapy for Enterobacteriaceae, such as Escherichia coli, and Klebsiella (e.g. Klebs. pneumoniae and Klebs. oxytoca) are antimicrobial drugs that are expensive due to prevailing patent protection. The replacement of such “ethical” drugs by a combination of “generic” antibiotics is thought to be beneficial from a therapeutic perspective as well as financial/economic perspective in times where governments are seeking to reduce the cost of healthcare.

The combinations of the present invention may be used to treat infections associated with any of the above-mentioned bacterial organisms, and in particular they may be used for killing multiplying and/or clinically latent microorganisms associated with such an infection, e.g. a Gram-negative bacterial infection.

Particular conditions which may be treated using the combination of the present invention include those which are caused by Gram-negative bacteria such as abscesses, asthma, bacilliary dysentry, bacterial conjunctivitis, bacterial keratitis, bacterial vaginosis, bone and joint infections, bronchitis (acute or chronic), brucellosis, burn wounds, cat scratch fever, cellulitis, chancroid, cholangitis, cholecystitis, cystic fibrosis, cystitis, nephritis, diffuse panbronchiolitis, dental caries, diseases of the upper respiratory tract, empymea, endocarditis, endometritis, enteric fever, enteritis, epididymitis, epiglottitis, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leptospirosis, listeriosis, liver abscesses, Lyme disease, lymphogranuloma venerium, mastitis, mastoiditis, meningitis and infections of the nervous system, non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, pleural effusion, pneumonia, postoperative wound infections, postoperative gas gangrene, prostatitis, pseudo-membranous colitis, psittacosis, pyelonephritis, Q fever, rat-bite fever, Ritter's disease, salmonellosis, salpingitis, septic arthritis, septic infections, septicameia, systemic infections, tonsillitis, trachoma, typhoid, urethritis, urinary tract infections, wound infections; or infections with, Escherichia coli, Klebs. pneumoniae, Klebs. oxytoca, Pr. mirabilis, Pr. rettgeri, Pr. vulgaris, Haemophilis influenzae, Enterococcus faecalis, Enterococcus faecium, and Enterobacter cloacae.

In one embodiment the combinations of the invention are used to treat urinary tract infections.

It will be appreciated that references herein to “treatment” extend to prophylaxis as well as the treatment of established diseases or symptoms.

As used herein the term “pharmaceutically acceptable derivative” means:
(a) pharmaceutically acceptable salts; and/or
(b) solvates (including hydrates).

Pharmaceutically acceptable salts of the compounds included in the combinations of the invention include suitable acid addition or base salts thereof. A review of suitable pharmaceutical salts may be found in Berge et al, J Pharm Sci, 66, 1-19 (1977).

Suitable acid addition salts include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulfonate salts (e.g. benzenesulfonate, methyl-, bromo- or chloro-benzenesulfonate, xylenesulfonate, methanesulfonate, ethanesulfonate, propanesulfonate, hydroxyethanesulfonate, 1- or 2-naphthalene-sulfonate or 1,5-naphthalenedisulfonate salts) or sulfate, pyrosulfate, bisulfate, sulfite, bisulfite, phosphate, monohydrogenphosphate, dihydrogenphosphate, metaphosphate, pyrophosphate or nitrate salts. Suitable base salts include metal salts, e.g. sodium, calcium, and amine salts.

For example, tetracycline hydrochloride, chlortetracycline hydrochloride, oxytetracycline hydrochloride, oxytetracycline hydrate, oxytetracycline dihydrate, demeclocycline hydrochloride, demeclocycline hydrochloride hydrate, meclocycline sulfosalicylate, methacycline hydrochloride, minocycline hydrochloride, doxycycline hyclate, doxycycline hydrochloride, doxycycline monohydrate, tigecycline hydrate, or sarecycline hydrochloride.

As used herein the term “prodrug” means the antimicrobial compound, wherein one or more groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo.

Examples of such modifications include ester formation (for example, any of those described above), wherein the reversion may be carried out be an esterase etc. Other such systems will be well known to those skilled in the art. Zidovudine is, for example, a prodrug that must be phosphorylated to its active 5′-triphosphate metabolite.

The invention includes the use of these pharmaceutically acceptable derivatives and prodrugs.

The invention also includes where appropriate all enantiomers and tautomers of the compounds. The skilled person will recognise compounds that possess optical properties (one or more chiral carbon atoms) or tautomeric characteristics. The corresponding enantiomers and/or tautomers may be isolated or prepared by methods known in the art.

Some of the compounds included in the combinations of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the compounds or pharmaceutically acceptable salts thereof. An isotopic variation or a pharmaceutically acceptable salt thereof is defined as one in which at least one atom is replaced by an atom having the same atomic number but an atomic mass different from the atomic mass usually found in nature. Examples of isotopes that can be incorporated include isotopes of hydrogen, carbon, nitrogen, oxygen, phosphorus, sulphur, fluorine and chlorine such as 2H, 3H, 13C, 14C, 15N, 17O, 18O, 31P, 32P, 35S, 18F and 36Cl, respectively. Certain isotopic variations, for example, those in which a radioactive isotope such as 3H or 14C is incorporated, are useful in drug and/or substrate tissue distribution studies. Tritiated, i.e., 3H, and carbon-14, i.e., 14C, isotopes are particularly preferred for their ease of preparation and detectability. Further, substitution with isotopes such as deuterium, i.e., 2H, may afford certain therapeutic advantages resulting from greater metabolic stability, for example, increased in vivo half-life or reduced dosage requirements and hence may be preferred in some circumstances. Isotopic variations can generally be prepared by conventional procedures using appropriate isotopic variations of suitable reagents.

The compounds for use in the combination of the present invention, including the pharmaceutically acceptable derivatives or prodrugs thereof, are commercially available and/or can be prepared by synthesis methods known in the art. Zidovudine, tetracycline, tetracycline hydrochloride, chlortetracycline, chlortetracycline hydrochloride, oxytetracycline, oxytetracycline hydrochloride, oxytetracycline hydrate, oxytetracycline dihydrate, demeclocycline hydrochloride, demeclocycline hydrochloride hydrate, meclocycline sulfosalicylate, methacycline hydrochloride, minocycline hydrochloride, doxycycline, doxycycline hyclate, doxycycline hydrochloride, doxycycline monohydrate, tigecycline, tigecycline hydrate, and sarecycline hydrochloride are for example available from Sigma-Aldrich®.

Other commercial suppliers are known in the art.

Zidovudine is 1-[(2R, 4S, 5S)-4-Azido-5-(hydroxymethyl)oxolan-2-yl]-5-methylpyrimidine-2,4-dione, and is available by prescription under the trade name Retrovir®. It is also known as 3′-azido-3′-deoxythymidine or “AZT” and has the following chemical structure:

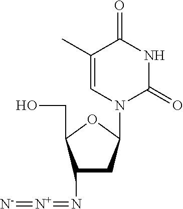 [SMILES: Cc1cn([C@H]2C[C@@H](N=[N+]=[N-])[C@@H](CO)O2)c(=O)[nH]c1=O]

Tetracycline is an antibiotic used to treat a number of infections; this includes acne, cholera, brucellosis, plague, malaria and syphilis. It is taken by mouth and has a broad spectrum of antibiotic action. It was originally made from bacteria of the Streptomyces type, and is sold under the brand name Sumycin among others, and available in tablet form as tetracycline hydrochloride. Its IUPAC chemical name is (4S,6S,12aS)-4-(dimethylamino)-1,4,4a,5,5a,6,11,12a-octahydro-3,6,10,12,12a-pentahydroxy-6-methyl-1,11-dioxonaphthacene-2-carboxamide.

The chemical structure of tetracycline is:

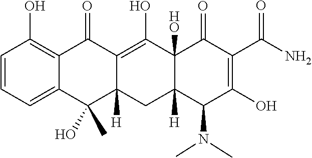 [SMILES: CN(C)[C@@H]1C(O)=C(C(N)=O)C(=O)[C@@]2(O)C(O)=C3C(=O)c4c(O)cccc4[C@@](C)(O)[C@H]3C[C@@H]12]

Chlortetracycline is tetracycline substituted by chlorine on the phenol ring; it was the first tetracycline to be identified and is available under the trade name Aureomycin. It has the IUPAC chemical name (4S,4aS,5aS,6S,12aR)-7-chloro-4-(dimethylamino)-1,6,10,11,12a-pentahydroxy-6-methyl-3,12-dioxo-4,4a,5,5a-tetrahydrotetracene-2-carboxamide, and the following chemical structure:

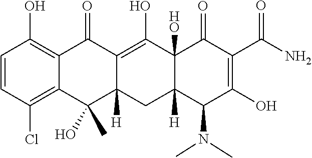 [SMILES: CN(C)[C@@H]1C(O)=C(C(N)=O)C(=O)[C@@]2(O)C(O)=C3C(=O)c4c(O)ccc(Cl)c4[C@@](C)(O)[C@H]3C[C@@H]12]

Oxytetracycline was the second of the broad-spectrum tetracycline group of antibiotics to be discovered. It is used to treat infections caused by Chlamydia, e.g. the chest infection psittacosis, the eye infection trachoma, and the genital infection urethritis, and infections caused by Mycoplasma organisms, e.g. pneumonia. Oxytetracycline is also used to treat acne and infections caused by Haemophilis influenzae, e.g. chronic bronchitis. It is available in tablet form as oxytetracycline dihydrate, or as an ointment (Terra-Cortril ointment) as oxytetracycline hydrochloride in combination with hydrocortisone. It has the IUPAC chemical name (4S,4aR,5S,5aR,6S,12aS)-4-(dimethylamino)-3,5,6,10,11,12a-hexahydroxy-6-methyl-1,12-dioxo-4,4a,5,5a,6,12,12a-octahydrotetracene-2-carboxamide, and the following chemical structure:

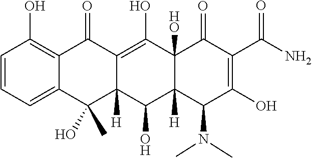 [SMILES: CN(C)[C@@H]1C(O)=C(C(N)=O)C(=O)[C@@]2(O)C(O)=C3C(=O)c4c(O)cccc4[C@@](C)(O)[C@H]3[C@H](O)[C@@H]12]

Demeclocycline is available under the brand names Declomycin, Declostatin, Ledermycin, Bioterciclin, Deganol, Detclo, Detravis, Meciclin, Mexocine and Clortetrin. It is a tetracycline antibiotic which was derived from a mutant strain of Streptomyces aureofaciens. Demeclocycline is indicated for the treatment of various types of bacterial infections, for example, Lyme disease, acne, and bronchitis. Its IUPAC chemical name is (4S,4aS,5aS,6S,12aS)-7-chlor-4-(dimethylamino)-3,6,10,12,12a-pentahydroxy-1,11-dioxo-1,4,4a,5,5a,6,11,12a-octahydro-2-tetracencarboxamide, and it has the following structure:

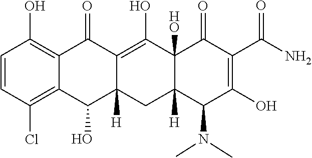 [SMILES: CN(C)[C@@H]1C(O)=C(C(N)=O)C(=O)[C@@]2(O)C(O)=C3C(=O)c4c(O)ccc(Cl)c4[C@@H](O)[C@H]3C[C@@H]12]

Lymecycline is a broad-spectrum antibiotic marketed by Galderma, which is used to treat a range of infections. Lymecycline is available in capsule form and marketed under various brand names including Tetralysal. Its IUPAC name is (2S)-6-[[[(4S,4aS,5aS,6S,12aR)-4-(dimethylamino)-1,6,10,11,12a-pentahydroxy-6-methyl-3,12-dioxo-4,4a,5,5a-tetrahydrotetracene-2-carbonyl]amino]methylamino]-2-aminohexanoic acid, and it has the following structure:

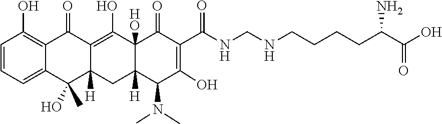 [SMILES: CN(C)[C@@H]1C(O)=C(C(=O)NCNCCCC[C@H](N)C(=O)O)C(=O)[C@@]2(O)C(O)=C3C(=O)c4c(O)cccc4[C@@](C)(O)[C@H]3C[C@@H]12]

Meclocycline is a topical tetracycline antibiotic. It has the IUPAC name (4S,4aR,5S,5aR,12aR)-7-chloro-4-(dimethylamino)-1,5,10,11,12a-pentahydroxy-6-methylidene-3,12-dioxo-4,4a,5,5a-tetrahydrotetracene-2-carboxamide and the following chemical structure:

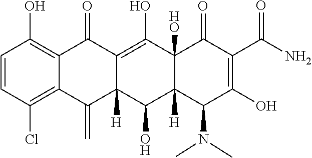 [SMILES: C=C1c2c(Cl)ccc(O)c2C(=O)C2=C(O)[C@]3(O)C(=O)C(C(N)=O)=C(O)[C@@H](N(C)C)[C@@H]3[C@@H](O)[C@H]12]

Methacycline or methacycline is used as a precursor in the industrial synthesis of doxycline hydrate but is a tetracycline antibiotic in its own right. It is available under the brand names of Esarondil, Physiomycine, and Rotilen, among others. The IUPAC name is (4S,4aR,5S,5aR,12aR)-4-(dimethylamino)-1,5,10,11,12a-pentahydroxy-6-methylidene-3,12-dioxo-4,4a,5,5a-tetrahydrotetracene-2-carboxamide, and the chemical structure is:

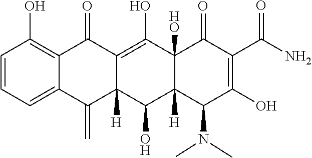 [SMILES: C=C1c2cccc(O)c2C(=O)C2=C(O)[C@]3(O)C(=O)C(C(N)=O)=C(O)[C@@H](N(C)C)[C@@H]3[C@@H](O)[C@H]12]

Minocycline, sold under the brand name Minocin among others, is a tetracycline antibiotic used to treat a number of bacterial infections such as pneumonia. It is also used for the treatment of acne and rheumatoid arthritis, along with other skin infections such as MRSA and Lyme disease. Minocycline is a tetracycline analogue having a dimethylamino group at position 7 and lacking the methyl and hydroxy groups at position 5. The IUPAC name of minocycline is (4S,4aS,5aR,12aR)-4,7-bis(dimethylamino)-1,10,11,12a-tetrahydroxy-3,12-dioxo-4a,5,5a,6-tetrahydro-4H-tetracene-2-carboxamide, and it has the following chemical structure:

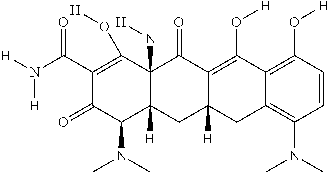 [SMILES: CN(C)c1ccc(O)c2c1C[C@@H]1C[C@@H]3[C@@H](N(C)C)C(=O)C(C(N)=O)=C(O)[C@]3(N)C(=O)C1=C2O]

Rolitetracycline is a tetracycline antibiotic which is a N-Mannich base prodrug that is prepared from tetracycline by condensation with pyrrolidine and formaldehyde. It is available under the brand name Reverin or Synterin and has the IUPAC name of (2Z,4S,4aS,5aS,6S,12aS)-4-Dimethylamino-6,10,11,12a-tetrahydroxy-2-[hydroxy-(pyrrolidin-1-ylmethylamino)methylidene]-6-methyl-4,4a,5,5a-tetrahydrotetracene-1,3,12-trione, and the following chemical structure:

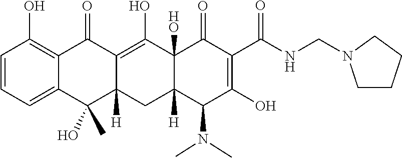 [SMILES: CN(C)[C@@H]1C(O)=C(C(=O)NCN2CCCC2)C(=O)[C@@]2(O)C(O)=C3C(=O)c4c(O)cccc4[C@@](C)(O)[C@H]3C[C@@H]12]

Doxycycline is an antibiotic used in the treatment of infections caused by bacteria and certain parasites. It is used to treat bacterial pneumonia, acne, Chlamydia infections, early Lyme disease, cholera and syphilis, amongst many other infections. It is available under the brand names of Doryx, Doxyhexal, Doxylin among others. It is tetracycline in which the 5beta hydrogen is replaced by a hydroxy group, while the 6alpha-hydroxy group is replaced by hydrogen. The IUPAC name of doxycycline is (4S,4aR,5S,5aR,6R,12aR)-4-(dimethylamino)-1,5,10,11,12a-pentahydroxy-6-methyl-3,12-dioxo-4a,5,5a,6-tetrahydro-4H-tetracene-2-carboxamide, and its chemical structure is:

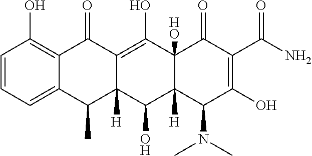 [SMILES: C[C@H]1c2cccc(O)c2C(=O)C2=C(O)[C@]3(O)C(=O)C(C(N)=O)=C(O)[C@@H](N(C)C)[C@@H]3[C@@H](O)[C@@H]21]

Tigecycline is an intravenously administered antibiotic for a number of bacterial infections. It is marketed by Pfizer under the brand name Tygacil, and used to treat different kinds of bacterial infections, including complicated skin and structure infections, complicated intra-abdominal infections and community-acquired bacterial pneumonia. It has the IUPAC name of (4S,4aS,5aR,12aR)-9-[[2-(tert-butylamino)acetyl]amino]-4,7-bis(dimethylamino)-1,10,11,12a-tetrahydroxy-3,12-dioxo-4a,5,5a,6-tetrahydro-4H-tetracene-2-carboxamide and the following chemical structure:

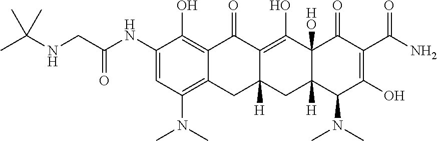 [SMILES: CN(C)c1cc(NC(=O)CNC(C)(C)C)c(O)c2c1C[C@H]1C[C@H]3[C@H](N(C)C)C(O)=C(C(N)=O)C(=O)[C@@]3(O)C(O)=C1C2=O]

Eravacycline is known under the brand name of Xerava (Tetraphase Pharmaceuticals) and is a parenterally administered synthetic halogenated tetracycline class antibiotic. It has a broad spectrum of activity against a variety of Gram-positive and Gram-negative bacteria including multi-drug resistant strains such as MRSA and carbapenem-resistant Enterobacteriaceae. The IUPAC name of eravacycline is (4S,4aS,5aR,12aR)-4-(dimethylamino)-7-fluoro-1,10,11,12a-tetrahydroxy-3,12-dioxo-9-[(2-pyrrolidin-1-ylacetyl)amino]-4a,5,5a,6-tetrahydro-4H-tetracene-2-carboxamide and it has the following chemical structure:

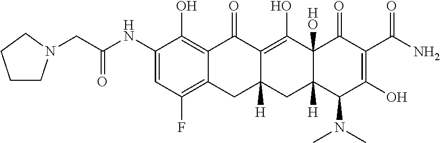 [SMILES: CN(C)[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

Sarecycline (trade name Seysara) has been approved by the FDA for the treatment of moderate to severe acne vulgaris, and is an orally administered tetracycline antibiotic. It has the IUPAC name of (4S,4aS,5aR,12aR)-4-(dimethylamino)-1,10,11,12a-tetrahydroxy-7-[[methoxy(methyl)amino]methyl]-3,12-dioxo-4a,5,5a,6-tetrahydro-4H-tetracene-2-carboxamide, and the following chemical structure:

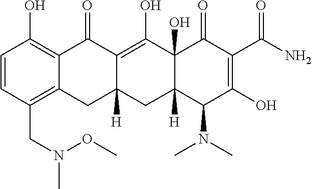 [SMILES: CON(C)Cc1ccc(O)c2c1C[C@H]1C[C@H]3[C@H](N(C)C)C(O)=C(C(N)=O)C(=O)[C@@]3(O)C(O)=C1C2=O]

Omadacycline is a broad spectrum antibiotic belonging to the aminomethylcycline subclass of tetracycline antibiotics. Both omadacycline and sarecycline are third generation tetracycline compounds. Omadacycline is known under the trade name of Nuzyra, and is used orally to treat moderate-to-severe infections including community-acquired pneumonia and acute bacterial skin and skin structure infections. The IUPAC name of omadacycline is (4S,4aS,5aR,12aR)-4,7-bis(dimethylamino)-9-[(2,2-dimethylpropylamino)methyl]-1,10,11,12a-tetrahydroxy-3,12-dioxo-4a,5,5a,6-tetrahydro-4H-tetracene-2-carboxamide, and it has the following chemical structure:

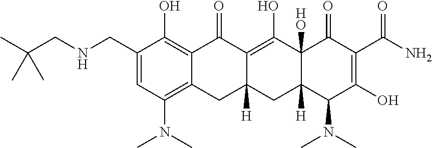 [SMILES: CN(C)c1cc(CNCC(C)(C)C)c(O)c2c1C[C@H]1C[C@H]3[C@H](N(C)C)C(O)=C(C(N)=O)C(=O)[C@@]3(O)C(O)=C1C2=O]

In various embodiments of the invention, the tetracycline antibiotic is selected from the group consisting of tetracycline, chlortetracycline, oxytetracycline, demeclocycline, lymecycline, meclocycline, methacycline, minocycline, rolitetracycline, doxycycline, tigecycline, eravacycline, sarecycline, omadacycline, and pharmaceutically acceptable derivatives and prodrugs thereof.

In preferred embodiments, the tetracycline antibiotic is selected from the group consisting of tetracycline, chlortetracycline, oxytetracycline, demeclocycline, meclocycline, methacycline, doxycycline, and pharmaceutically acceptable derivatives and prodrugs thereof. In more preferred embodiments, the tetracycline antibiotic is selected from the group consisting of tetracycline, chlortetracycline, oxytetracycline, demeclocycline, doxycycline, and pharmaceutically acceptable derivatives and prodrugs thereof. In most preferred embodiments, the tetracycline antibiotic is selected from the group consisting of tetracycline, chlortetracycline, oxytetracycline, doxycycline, and pharmaceutically acceptable derivatives and prodrugs thereof. For example, the tetracycline antibiotic may be doxycycline or a pharmaceutically acceptable derivative or prodrug thereof.

Compounds for use according to the invention may be administered as the raw material but are preferably provided in the form of pharmaceutical compositions. The compounds may be used either as separate formulations or as a single combined formulation. When combined in the same formulation it will be appreciated that the two compounds must be stable and compatible with each other and the other components of the formulation.

Formulations of the invention include those suitable for oral, parenteral (including subcutaneous e.g. by injection or by depot tablet, intrathecal, intramuscular e.g. by depot and intravenous), and rectal or in a form suitable for administration by inhalation or insufflation administration. The most suitable route of administration may depend upon the condition and disorder of the patient. Preferably, the compositions of the invention are formulated for oral administration.

The formulations may conveniently be presented in unit dosage form and may be prepared by any of the methods well known in the art of pharmacy e.g. as described in “Remington: The Science and Practice of Pharmacy”, Lippincott Williams and Wilkins, 21^{st} Edition, (2005). Suitable methods include the step of bringing into association to active ingredients with a carrier which constitutes one or more excipients. In general, formulations are prepared by uniformly and intimately bringing into association the active ingredients with liquid carriers or finely divided solid carriers or both and then, if necessary, shaping the product into the desired formulation. It will be appreciated that when the two active ingredients are administered independently, each may be administered by a different means.

When formulated with excipients, the active ingredients may be present in a concentration from 0.1 to 99.5% (such as from 0.5 to 95%) by weight of the total mixture; conveniently from 30 to 95% for tablets and capsules and 0.01 to 50% (such as from 3 to 50%) for liquid preparations.

Formulations suitable for oral administration may be presented as discrete units such as capsules, cachets or tablets (e.g. chewable tablets in particular for paediatric administration), each containing a predetermined amount of active ingredient; as powder or granules; as a solution or suspension in an aqueous liquid or non-aqueous liquid; or as an oil-in-water liquid emulsion or water-in-oil liquid emulsion. The active ingredients may also be presented a bolus, electuary or paste.

A tablet may be made by compression or molding, optionally with one or more excipients. Compressed tablets may be prepared by compressing in a suitable machine the active ingredient in a free-flowing form such as a powder or granules, optionally mixed with other conventional excipients such as binding agents (e.g. syrup, acacia, gelatin, sorbitol, tragacanth, mucilage of starch, polyvinylpyrrolidone and/or hydroxymethyl cellulose), fillers (e.g. lactose, sugar, microcrystalline cellulose, maize-starch, calcium phosphate and/or sorbitol), lubricants (e.g. magnesium stearate, stearic acid, talc, polyethylene glycol and/or silica), disintegrants (e.g. potato starch, croscarmellose sodium and/or sodium starch glycolate) and wetting agents (e.g. sodium lauryl sulphate). Molded tablets may be made by molding in a suitable machine a mixture of the powdered active ingredient with an inert liquid diluent. The tablets may be optionally coated or scored and may be formulated so as to provide controlled release (e.g. delayed, sustained, or pulsed release, or a combination of immediate release and controlled release) of the active ingredients.

Alternatively, the active ingredients may be incorporated into oral liquid preparations such as aqueous or oily suspensions, solutions, emulsions, syrups or elixirs. Formulations containing the active ingredients may also be presented as a dry product for constitution with water or another suitable vehicle before use.

Such liquid preparations may contain conventional additives such as suspending agents (e.g. sorbitol syrup, methyl cellulose, glucose/sugar syrup, gelatin, hydroxymethyl cellulose, carboxymethyl cellulose, aluminium stearate gel and/or hydrogenated edible fats), emulsifying agents (e.g. lecithin, sorbitan mono-oleate and/or acacia), non-aqueous vehicles (e.g. edible oils, such as almond oil, fractionated coconut oil, oily esters, propylene glycol and/or ethyl alcohol), and preservatives (e.g. methyl or propyl p-hydroxybenzoates and/or sorbic acid).

Combinations for use according to the invention may be presented in a pack or dispenser device which may contain one or more unit dosage forms containing the active ingredients. The pack may, e.g. comprise metal or plastic foil, such as a blister pack. Where the compositions are intended for administration as two separate compositions these may be presented in the form of a twin pack.

Pharmaceutical compositions may also be prescribed to the patient in “patient packs” containing the whole course of treatment in a single package, usually a blister pack. Patient packs have an advantage over traditional prescriptions, where a pharmacist divides a patients' supply of a pharmaceutical from a bulk supply, in that the patient always has access to the package insert contained in the patient pack, normally missing in traditional prescriptions. The inclusion of the package insert has been shown to improve patient compliance with the physician's instructions.

The administration of the combination of the invention by means of a single patient pack, or patient packs of each composition, including a package insert directing the patient to the correct use of the invention is a desirable feature of this invention.

According to a further embodiment of the present invention there is provided a patient pack comprising at least one active of the combination according to the invention and an information insert containing directions on the use of the combination of the invention. In another embodiment of the invention, there is provided a double pack comprising in association for separate administration, an antimicrobial agent, preferably having biological activity against clinically latent microorganisms, and one or more of the compounds disclosed herein preferably having biological activity against clinically latent microorganisms.

The amount of active ingredients required for use in treatment will vary with the nature of the condition being treated and the age and condition of the patient, and will ultimately be at the discretion of the attendant physician. In general however, doses employed for adult human treatment will typically be in the range of 0.02 to 5000 mg per day, preferably 1 to 1500 mg per day. The desired dose may conveniently be presented in a single dose or as divided doses administered at appropriate intervals, e.g. as two, three or more sub-doses per day.

Suitable dosages and formulations for the administration of zidovudine are described in the product label for Retrovir® oral solution or capsules which can be found at http://www.medicines.org.uk/emc/medicine/12444/SPC/Retrovir+250 mg+Capsules/.

Suitable dosages and formulations for the administration of the tetracycline antibiotic are described in the product labels for e.g. tetracycline tablets, oxytetracycline tablets, or ointment including oxytetracycline hydrochloride, demeclocycline hydrochloride capsules, lymecycline capsules, minocycline hydrochloride capsules or tablets, doxycycline hyclate capsules or tablets, doxycycline monohydrate capsules or tablets, or tigecycline powder for solution for infusion. Such labels can be readily found for the skilled person, including by searching for the tetracycline antibiotic at https://www.medicines.org.uk/emc/browse-inqredients#

This information would therefore be readily obtained and understood by the person skilled in the art.

### Biological Tests

Test procedures that may be employed to determine the biological (e.g. bactericidal or antimicrobial) activity of the active ingredients include those known to persons skilled in the art for determining:
(a) bactericidal activity against clinically latent bacteria; and
(b) antimicrobial activity against log phase bacteria.

In relation to (a) above, methods for determining activity against clinically latent bacteria include a determination, under conditions known to those skilled in the art (such as those described in Nature Reviews, Drug Discovery 1, 895-910 (2002), the disclosures of which are hereby incorporated by reference), of Minimum Stationary-cidal Concentration (“MSC”) or Minimum Dormicidal Concentration (“MDC”) for a test compound.

By way of example, WO2000028074 describes a suitable method of screening compounds to determine their ability to kill clinically latent microorganisms. A typical method may include the following steps:
(1) growing a bacterial culture to stationary phase;
(2) treating the stationery phase culture with one or more antimicrobial agents at a concentration and or time sufficient to kill growing bacteria, thereby selecting a phenotypically resistant sub-population;
(3) incubating a sample of the phenotypically resistant subpopulation with one or more test compounds or agents; and
(4) assessing any antimicrobial effects against the phenotypically resistant subpopulation.

According to this method, the phenotypically resistant sub-population may be seen as representative of clinically latent bacteria which remain metabolically active in vivo and which can result in relapse or onset of disease.

In relation to (b) above, methods for determining activity against log phase bacteria include a determination, under standard conditions (i.e. conditions known to those skilled in the art, such as those described in WO 2005014585, the disclosures of which document are hereby incorporated by reference), of Minimum Inhibitory Concentration (“MIC”) or Minimum Bactericidal Concentration (“MBC”) for a test compound. Specific examples of such methods are described below.

### Examples

### In Vitro Synergistic Effect of Zidovudine (AZT) and Doxycycline

The chequerboard method used in Example 1 followed the protocols detailed in Antimicrob Chemo (2013) 68, 374-384. Zidovudine and doxycycline were obtained from commercially available sources. The bacteria used were BAA2469 NDM-1 Escherichia coli, BAA2470 NDM-1 Klebsiella pneumoniae, BAA2471 NDM-1 Escherichia coli, BAA2472 NDM-1 Klebsiella pneumoniae, and NCTC13443 NDM-1 Klebsiella pneumoniae. All strains were obtained from a commercial source and log phase growth of the bacteria was carried out using methods known in the art.

The effects of the combination of the present invention were examined by calculating the fractional inhibitory concentration index (FICI) of each combination, as follows:

(MIC of drug A,tested in combination)/(MIC of drug A,tested alone)+(MIC of drug B,tested in combination)/(MIC of drug B,tested alone).

The interaction of the combination was defined as showing synergy if the FICI was 50.5, no interaction if the FICI was >0.5 but <4.0 and antagonism if the FICI was >4.0.

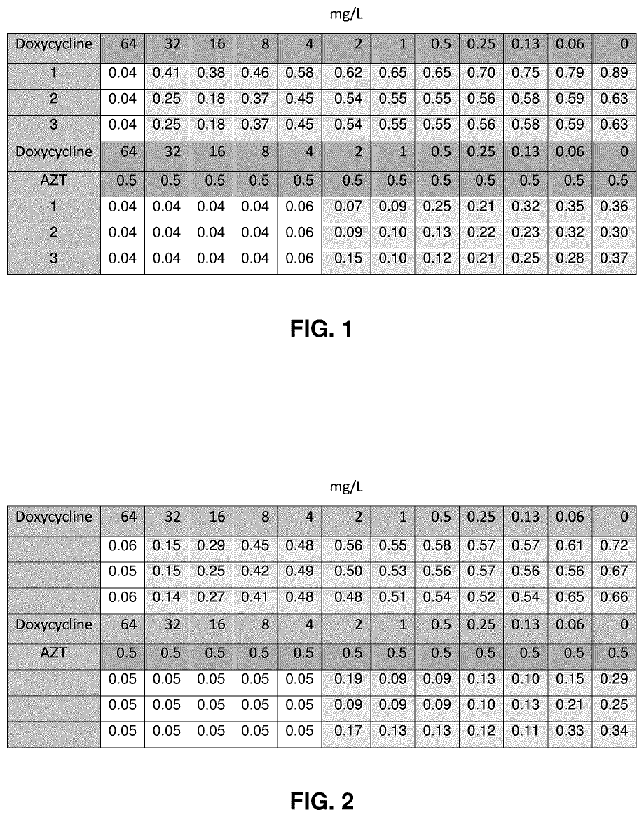

FIG. 1 shows the results on BAA2469 NDM-1 Escherichia coli.

FIG. 2 shows the results on BAA2470 NDM-1 Klebsiella pneumoniae.

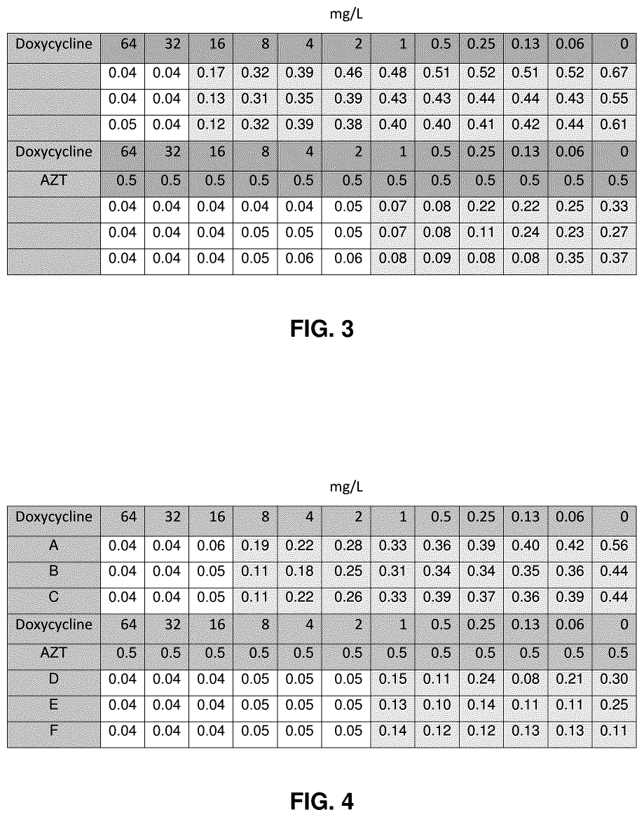

FIG. 3 shows the results on BAA2471 NDM-1 Escherichia coli.

FIG. 4 shows the results on BAA2472 NDM-1 Klebsiella pneumoniae.

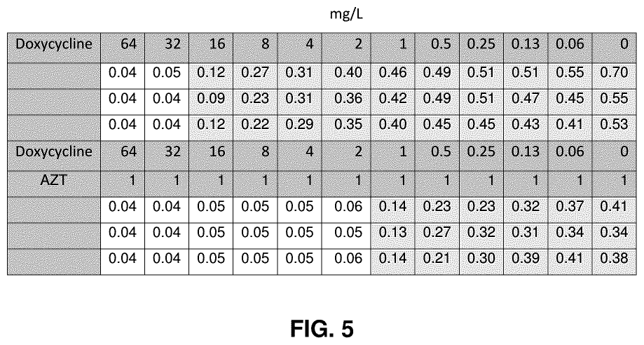

FIG. 5 shows the results on NCTC13443 NDM-1 Klebsiella pneumoniae.

TABLE 1
The reduction of doxycycline MIC in combination with AZT

| | MIC | | MIC fold |
|---|---|---|---|
| | Doxycycline | Doxycycline + AZT | reduction |
| BAA2469 | 64 | 4 | 16 |
| BAA2470 | 64 | 4 | 16 |
| BAA2471 | 32 | 2 | 16 |
| BAA2472 | 16 | 2 | 8 |
| NCTC13443 | 32 | 2 | 16 |

A MIC reduction of ≥4 fold indicates a synergistic effect. For doxycycline+AZT, synergistic effects were shown against all the strains tested, for example for strain BAA2469 NDM-1 E. coli or BAA2470 NDM-1 K. pneumoniae, doxycycline MIC against the strain was 64 mg/L, but in combination with 0.5 mg/L AZT, its MIC reduced to 4 showing a 16-fold reduction for both strains.

1. A method of treating a microbial infection, the method comprising administering to a subject a combination comprising zidovudine or a pharmaceutically acceptable derivative thereof and a tetracycline antibiotic or a pharmaceutically acceptable derivative or prodrug thereof.

2. The method of claim 1 wherein the combination kills multiplying microorganisms associated with the microbial infection.

3. The method of claim 2, wherein the microbial infection is a bacterial infection and the microorganisms are bacteria.

4. The method of claim 3, wherein the infection is a gram-negative bacterial infection.

5. The method of claim 1, wherein the microbial infection is a urinary tract infection.

6. The method of claim 3, wherein the bacterial infection is caused by Enterobacteriaceae.

7. The method of claim 1, wherein the microbial infection is caused by a drug-resistant strain of bacteria.

8. The method of claim 1, wherein the tetracycline antibiotic is selected from the group consisting of tetracycline, chlortetracycline, oxytetracycline, demeclocycline, lymecycline, meclocycline, methacycline, minocycline, rolitetracycline, doxycycline, tigecycline, eravacycline, sarecycline, omadacycline, and pharmaceutically acceptable derivatives and prodrugs thereof.

9. The method of claim 8, wherein the tetracycline antibiotic is selected from the group consisting of tetracycline, chlortetracycline, oxytetracycline, demeclocycline, meclocycline, methacycline, doxycycline, and pharmaceutically acceptable derivatives and prodrugs thereof.

10. The method of claim 9, wherein the tetracycline antibiotic is selected from the group consisting of tetracycline, chlortetracycline, oxytetracycline, demeclocycline, doxycycline, and pharmaceutically acceptable derivatives and prodrugs thereof.

11. The method of claim 10, wherein the tetracycline antibiotic is selected from the group consisting of tetracycline, chlortetracycline, oxytetracycline, doxycycline, and pharmaceutically acceptable derivatives and prodrugs thereof.

12. The method of claim 11, wherein the tetracycline antibiotic is doxycycline or a pharmaceutically acceptable derivative or prodrug thereof.

13. A pharmaceutical composition comprising zidovudine or a pharmaceutically acceptable derivative thereof in combination with a tetracycline antibiotic or a pharmaceutically acceptable derivative or prodrug thereof, and a pharmaceutically acceptable adjuvant, diluent or carrier, for use in the treatment of a microbial infection.

14. The pharmaceutical composition according to claim 13, wherein the tetracycline antibiotic is selected from the group consisting of tetracycline, chlortetracycline, oxytetracycline, demeclocycline, lymecycline, meclocycline, methacycline, minocycline, rolitetracycline, doxycycline, tigecycline, eravacycline, sarecycline, omadacycline, and pharmaceutically acceptable derivatives and prodrugs thereof.

15. The pharmaceutical composition according to claim 14, wherein the tetracycline antibiotic is selected from the group consisting of tetracycline, chlortetracycline, oxytetracycline, demeclocycline, meclocycline, methacycline, doxycycline, and pharmaceutically acceptable derivatives and prodrugs thereof.

16. The pharmaceutical composition according to claim 15, wherein the tetracycline antibiotic is selected from the group consisting of tetracycline, chlortetracycline, oxytetracycline, demeclocycline, doxycycline, and pharmaceutically acceptable derivatives and prodrugs thereof.

17. The pharmaceutical composition according to claim 16, wherein the tetracycline antibiotic is selected from the group consisting of tetracycline, chlortetracycline, oxytetracycline, doxycycline, and pharmaceutically acceptable derivatives and prodrugs thereof, preferably wherein the tetracycline antibiotic is doxycycline or a pharmaceutically acceptable derivative or prodrug thereof.

18. A product comprising zidovudine or a pharmaceutically acceptable derivative thereof and a tetracycline antibiotic or a pharmaceutically acceptable derivative or prodrug thereof, as a combined preparation for simultaneous, separate or sequential use in treating a microbial infection.

19. The product according to claim 18, wherein the tetracycline antibiotic is selected from the group consisting of tetracycline, chlortetracycline, oxytetracycline, demeclocycline, lymecycline, meclocycline, methacycline, minocycline, rolitetracycline, doxycycline, tigecycline, eravacycline, sarecycline, omadacycline, and pharmaceutically acceptable derivatives and prodrugs thereof.

20. The product according to claim 19, wherein the tetracycline antibiotic is selected from the group consisting of tetracycline, chlortetracycline, oxytetracycline, demeclocycline, meclocycline, methacycline, doxycycline, and pharmaceutically acceptable derivatives and prodrugs thereof.

21. The product according to claim 20, wherein the tetracycline antibiotic is selected from the group consisting of tetracycline, chlortetracycline, oxytetracycline, demeclocycline, doxycycline, and pharmaceutically acceptable derivatives and prodrugs thereof.

22. The product according to claim 21, wherein the tetracycline antibiotic is selected from the group consisting of tetracycline, chlortetracycline, oxytetracycline, doxycycline, and pharmaceutically acceptable derivatives and prodrugs thereof.

23. The product according to claim 22, wherein the tetracycline antibiotic is doxycycline or a pharmaceutically acceptable derivative or prodrug thereof.
